SEQUENCE LISTING (11 sequences)

SEQ ID NO: 1 - DNA, 21 bp, Artificial sequence
  Synthetic oligonucleotide primer or probe
ttcaactgaagcaacaccttc

SEQ ID NO: 2 - DNA, 25 bp, Artificial sequence
  Synthetic oligonucleotide primer or probe
cacttgtattaaccgcttgattaac

SEQ ID NO: 3 - DNA, 25 bp, Artificial sequence
  Synthetic oligonucleotide primer or probe
tacttggattaaccgcttgactaac

SEQ ID NO: 4 - DNA, 21 bp, Artificial sequence
  Synthetic oligonucleotide primer or probe
aactccagaacgtgattctga

SEQ ID NO: 5 - DNA, 21 bp, Artificial sequence
  Synthetic oligonucleotide primer or probe
tgatttgaccacgttcaacac

SEQ ID NO: 6 - DNA, 27 bp, Artificial sequence
  Synthetic oligonucleotide primer or probe
tgaatcagctccacagagtacagatgc

SEQ ID NO: 7 - DNA, 25 bp, Artificial sequence
  Synthetic oligonucleotide primer or probe
cattcatgatgccagttgaggacgt

SEQ ID NO: 8 - DNA, 3090 bp, Staphylococcus aureus
  misc_feature: aureus strain MRSA252
atgaatatgaagaaacaagaaaaacacgcaattcgtaaaaaatcgattggcgtggcttcagtgcttgtaggtacgttaatcggttttggactactcagcagtaaagaagcagatgcaagtgaaaatagtatgacacaaacggaaaatacgagtaatgagagcaaaagtaatgatccaagtagcgttaatgctgcacctaaaacagacaacacaaacgtgagtgattctaatacaacgacaaacactaatagtgacgaaacgaatgtagcgcaaaatccagcacaacaggaaacgacacaatcagcatcaacaaatgcaactacagaagaaacaccggtaactggtgaagttactactacggcaacgaatcaagctaatacaccggcaacaactcaatcaagcaatacaaatgcggaagaatcagtgaatcaaacaagtaatgaaacgacttctaatgatactaatacagtatcatctgtaaattcacctcaaaattctacaaatgcggaaaatgtttcaacaacgcaagacatttcaactgaagcaacaccttcaaacaatgaatcagctccacagagtacagatgcaagtaataaagatgtagttaatcaagcggttaatacaagtgcgcctagaatgagagcatttagtttagcggctgtagctgcagatgcaccggctgctggcaaagatattacgaatcagttgacgaatgtgacagttggtattgactctggagatacagtttatccgcaccaagcaggctatgtcaaactgaattatgggttctcagtaccaaatgaggctgttcaaggtgacacattcaaaataactgtgcccaaagaattaaacttaaatggtgtaacttcaactgctaaagtgccaccaattatggccggagatcaagtattggcaaatggtgtaatcgatagtgatggtaatgttatttatacatttacagactatgtaaatactaaagatgatgttaaagcaactttgaccatgcccgcttatattgaccctgaaaatgttacaaagacaggtaatgtgacattggctactggcataggtagtacaacagcaaacaaaacagtattagtagattatgaaaaatatggtaagttttataacttatctattaaaggtacaattgaccaaatcgataaaacaaataatacgtatcgtcagacaatttatgtcaatccaagtggagataacgttattgcgccggttttaacaggtaatttaaaaccaaatacggatagtaatgcattaatagatgcacaaaatactagtattaaagtatataaagttgataatgcatcagacttgtctgaaagttattatgtgaatccagataactttgaagatgtcactgatagtgtgaatattacattcccaaatccaaatcaatataaagtagagttcaatacgcctgatgatcaaataacaacaccatatattgtagttgttaatgggcatattgatcctaatagtaaaggtgatttagctttacgttcaactttatatggatataattcgaatataatttggcgatcaatgtcatgggataatgaagtagcatttaataacggatcaggttctggtgacggtatcgataaacctgttgttcctgaacaacctgatgagccgggtgaaattgaaccaattccagaggattcagattctgacccaggttcagatagtggttcagattctggcagcgattctaattcagatagcggttcagattcgggtagtgattctacatcagatagtggttcagattcagcgagcgattcagattcagcaagtgattcagattcagcgagtgattcagattcagcaagtgattcagattcagcgagtgattcagattcagcaagtgattcagattcagcgagtgattcagattcaacgagtgattcagattcaacgagtgattccgattcagcaagcgattcagattcagatagtgactcagactcagacagcgattcagattcagatagtgactcagactcagatagcgattcagattccgacagtgactcggattcagatagcgattccgactcagacagtgactcagattcagatagcgactcagactcagaaagcgactcagattcagacagcgattccgactcagatagtgattcagattcagacagcgactcagacagtgtttcagattcagacagtgactcagactcggatagtgaatcagactcagacagcgactcagattcagacagcgactcagactcggatagtgaatcagactcagacagtgactcagactcagacagcgactcagactcagatagcgactcagattcagatagcgattcagactcagacagcgactcagactcagatagcgattccgactcggatagcgactcagactcagatagcgattccgactcggatagcgattcagaatcagatagtgactcagattcagatagcgattccgactcagatagcgactcagattccgacagcgattcagattcagacagtgatttagactcagacagtgactcggattcagatagcgattccgactcagacagtgactcggattccgacagcgattcagattcagacagtgatttagactcagacagtgactcggattcagatagcgattccgactcagatagtgcctcagattcagatagcgactcagactcagacagcgattcagaatcagatagtgactcagattcagatagcgattccgactcagacagtgcctcagattcagatagtgactcggattcagcgagtgattccgattcagattcaacgagtgacacaggatcagataacgactcagactcagaaagtgattcgaatagcgattccgattcaggttctaacaataatgtagtgccgcctaattcacctaaaagtggtactaatgcttcaaataaaaatgaggctaaagaaagtaaagaaccattaccagatacaggttctgaagatgaagctaatacgtcactaatttggggattattagcatcattaggttcattactacttttcagaagaaaaaaagaaaataaagataagaaataa

SEQ ID NO: 9 - DNA, 1185 bp, Staphylococcus epidermidis
  misc_feature: ATCC 12228
atggcaaaagaaaaatttgatcgctcaaaagaacatgccaatattggtactatcggtcacgttgaccatggtaaaacaactttaacagctgctatcgcaactgtattagctaaaaatggtgacactgttgcacaatcatacgatatgattgacaacgctccagaagaaaaagaacgtggtattacaatcaatactgcacatatcgaataccaaactgacaaacgtcactatgctcacgttgactgcccaggacacgctgactatgttaaaaacatgatcactggtgcagctcaaatggacggcggtatcttagttgtatctgctgctgacggtccaatgccacaaactcgtgaacacatcttattatcacgtaacgttggtgtaccagcattagttgtattcttaaacaaagttgacatggtagacgacgaagaattattagaattagttgaaatggaagttcgtgacttattaagcgaatatgacttcccaggtgacgatgtacctgtaatcgctggttctgcattaaaagcattagaaggcgatgctgaatacgaacaaaaaatcttagacttaatgcaagcagttgatgattacattccaactccagaacgtgattctgacaaaccattcatgatgccagttgaggacgtattctcaatcactggtcgtggtactgttgctacaggccgtgttgaacgtggtcaaatcaaagttggtgaagaagttgaaatcatcggtatgcacgaaacttctaaaacaactgttactggtgtagaaatgttccgtaaattattagactacgctgaagctggtgacaacatcggtgctttattacgtggtgttgcacgtgaagacgtacaacgtggtcaagtattagctgctcctggttctattacaccacacacaaaattcaaagctgaagtatacgtattatctaaagatgaaggtggacgtcacactccattcttcactaactatcgcccacaattctatttccgtactactgacgtaactggtgttgtaaacttaccagaaggtacagaaatggttatgcctggcgacaacgttgaaatgacagttgaattaatcgctccaatcgctatcgaagacggaactcgtttctcaattcgtgaaggtggacgtactgttggatcaggcgttgtaactgaaatctttgaataa

SEQ ID NO: 10 - DNA, 95 bp, Staphylococcus aureus
ttcaactgaagcaacaccttcaaacaatgaatcagctccacagagtacagatgcaagtaataaagatgtagttaatcaagcggttaatacaagtg

SEQ ID NO: 11 - DNA, 110 bp, Staphylococcus sp.
aactccagaacgtgattctgacaaaccattcatgatgccagttgaggacgtattctcaatcactggtcgtggtactgttgctacaggccgtgttgaacgtggtcaaatca

(19) United States
(12) Patent Application Publication
(10) Pub. No.: US 2009/0191561 A1
(43) Pub. Date: Jul. 30, 2009

(54) METHODS AND OLIGONUCLEOTIDES FOR DETECTION OF MASTITIS CAUSING BACTERIA

(75) Inventors: Jani Holopainen, Helsinki (FI); Mikko Koskinen, Espoo (FI)

Correspondence Address: BIRCH STEWART KOLASCH & BIRCH, PO BOX 747, FALLS CHURCH, VA, 22040-0747, US

(73) Assignee: Finnzymes Oy, Espoo (FI)

(21) Appl. No.: 12/347,519

(22) Filed: Dec. 31, 2008

Related U.S. Application Data
(60) Provisional application No. 61/017,990, filed on Dec. 31, 2007.

(51) Int. Cl. C12Q 1/68; C07H 21/04

(52) U.S. Cl. 435/6; 536/24.33

(57) ABSTRACT

The present invention relates to the field of polymerase chain reaction (PCR) based diagnostic assays. More specifically, the present invention provides a PCR-method for detecting mastitis causing bacteria, particularly Staphylococcus aureus and coagulase negative staphylococci. The present invention also provides oligonucleotide primers and probes for use in said method.

This Nonprovisional application claims priority under 35 U.S.C. § 119(e) on U.S. Provisional Application Nos. 61/017,990 filed on Dec. 31, 2007, the entire contents of which is hereby incorporated by reference.

The present invention relates to the field of polymerase chain reaction (PCR) based diagnostic assays. More specifically, the present invention provides a PCR based method for detecting mastitis causing bacteria, particularly Staphylococcus aureus and coagulase negative staphylococci. The present invention also provides oligonucleotide primers and probes for use in said method.

### BACKGROUND OF THE INVENTION

Mastitis, i.e. inflammation of a cow's mammary gland, leads to a significant decrease in milk yield and quality and represents a major problem to the dairy industry world-wide. In acute mastitis, the udder is commonly infected by one primary pathogen. In many countries, staphylococci are the most common agents of mastitis: for example in Finland, in 2001, more than one-half of the intramammary infections were caused by either coagulase negative staphylococci (CNS) or Staphylococcus aureus (Pitkälä et al. 2004). In contrast to CNS and S. aureus responsible for human infections, the bovine mastitis-related staphylococci can often be successfully treated with beta-lactam antibiotics (e.g., penicillin). Resistance of CNS and S. aureus to beta-lactam antibiotics is caused by an enzyme known as beta-lactamase. The ‘blaZ’ gene is responsible for production of beta-lactamase and CNS and S. aureus harbouring the gene are penicillin resistant, while strains lacking the gene can be successfully treated with beta-lactam antibiotics.

Bacterial culturing on blood-esculin-agar remains the gold standard for diagnosing intra-mammary infections. When correctly performed, culturing can provide a very specific and sensitive means for mastitis pathogen testing, as well as testing their capability to produce beta-lactamase. However, culturing is notoriously slow, with laboratory turn-around times being routinely up-to 48 hours. Consequently, the current testing schemes often fail to guide the treatment of an animal. Instead, due to the long analyses times, antibiotic treatment is routinely started for cows exhibiting symptoms of an acute infection, prior to receiving the test results (Sandholm 1995). It follows, that in a significant proportion of all mastitis cases the treatment may be unnecessary either due to resistance of the pathogen to the primary choice of antibiotics (penicillin) or due to the fact that in some 20% of inflammations the infection is caused by something else than bacteria sequestered in the udder (Sandholm 1995). This practice leads to much unnecessary antibiotic use, as well as significant economical losses to the farmers, through added drug costs and discarded milk. PCR (polymerase chain reaction) assays and its derivatives, such as qPCR assays (quantitative polymerase chain reaction, also known as real-time polymerase chain reaction), aiming to amplify, identify and quantify species-specific nucleic acids have become widely used in routine microbial testing. PCR methods have potential to provide significant analysis time savings. Hence, PCR-based tests would seem to hold much promise for mastitis testing (see, e.g., Gillespie & Oliver 2005).

### Challenges in the Development of a PCR Method for Mastitis Testing

The most important mastitis pathogens include at least the following: Staphylococcus aureus, Coagulase Negative Staphylococci (CNS; a species group comprising over 30 genetically closely related but difficult-to-identify species), Streptococcus dysgalactiae, Streptococcus agalactiae, Streptococcus uberis, Escherichia coli, Enterococcus spp., Corynebacterium bovis, Arcanobacterium pyogenes, Klebsiella spp., Lactococcus lactis and Serratia spp.

In the development of PCR assays, one of the most important factors is to locate oligonucleotide sequences that enable reliable species-specific amplification, detection and quantification. It is of utmost importance that a given set of oligonucleotides, designed to amplify e.g., Staphylococcus aureus, does not cross-react with DNA originating from any other species possibly present in the sample matrix. Finding such sequences can be far from trivial, at least for the following reasons: 1) many of the species are relatively closely related, making it challenging to locate sequences that are unique for each species; 2) mastitis pathogen strains originating from a single species can be genetically diverged, making it difficult to locate sequences that would enable equally efficient amplification of all strains within a species: 3) the sample matrix in mastitis diagnostics is commonly raw milk, containing a host of PCR inhibitors. Hence, in order to effectively amplify pathogen DNA from all milk samples requires oligonucleotide design enabling high PCR efficiency (optimally as close to 100% as possible); 4) the number of pathogens causing mastitis is large (see above) and a mastitis testing method should optimally identify all of them. Having one PCR reaction per species can be cumbersome, since the number of samples tested is typically large. It would be optimal to detect multiple species within one reaction. In a PCR setting the most obvious alternative is ‘multiplex’ PCR amplification. In multiplex PCR, several oligonucleotide sets, each designed to amplify one species/species group, are included in the same reaction vessel and each oligonucleotide set is used to amplify its respective pathogen DNA during the same PCR reaction. Multiplex PCR presents a challenge for quantitation of the pathogen DNA (qPCR): the different amplicons compete for the same PCR reaction components (e.g. DNA polymerase and MgCl2) and this can compromise the quantitative nature of the reaction between and, especially, quantitative comparisons between samples.

The present inventors have now located DNA sequence regions that are well suited for specific and sensitive amplification and quantification of mastitis pathogens, particularly Staphylococcus aureus and coagulase negative staphylococci, using template DNA extracted from raw bovine milk. Optimal primers and quantitative PCR probes have been designed and validated for identification and quantification of the mastitis pathogens. A collection of hundreds of bacterial strains has been used in assessing the specificity of the oligonucleotide design.

Prior PCR assays for the detection of Staphylococcus aureus and Staphylococci are disclosed by Sakai et al, 2004, Maes et al, 2002 and Martineau et al, 2001 as well as in U.S. Pat. No. 5,702,895. Other prior art related to this particular technical field are: U.S. Pat. No. 5,849,488 disclosing a method for detecting presence of DNA sequences in a milk sample; U.S. Pat. No. 6,720,160 disclosing a method for simultaneous detection of plurality of different indicators of mastitis; and Riffon et al, 2001, disclosing development of a PCR-test for identification of major pathogens in bovine mastitis.

### DETAILED DESCRIPTION OF THE INVENTION

The present invention is directed to a method for determining the presence of mastitis causing bacteria in a milk sample comprising the steps of:

a) isolating nucleic acid from a milk sample or a sample derived from milk, preferably as described in Gillespie and Oliver 2005;
b) contacting the isolated nucleic acid obtained in step a) in a polymerase chain reaction mix with primers specifically hybridizing with the nucleic acid sequence of clumping factor gene of Staphylococcus aureus and primers specifically hybridizing with the nucleic acid sequence of elongation factor Tu gene of Staphylococcus spp.; and
c) performing a polymerase chain reaction with a reaction mix obtained from step b), wherein the detection of desired hybrids formed between said primers and said isolated nucleic acid indicates the presence of Staphylococcus aureus and/or Staphylococcus spp. in said sample. The desired hybrids are preferably detected by determining the presence of a PCR-product generated by extension of said primers while said primers are specifically hybridized to the isolated nucleic acid, i.e. said isolated nucleic acid is used as a template for the extension product. In other words, a polymerase chain reaction is performed with a reaction mix obtained from step b) so that the sequences of said clumping factor gene and/or elongation factor Tu gene are specifically amplified, if said sequences are present in the sample. Thereafter, the presence of the amplified nucleid acid sequences is detected, wherein the presence of amplified nucleic acid sequence from both clumping factor gene and elongation factor Tu gene is indicative of the presence of Staphylococcus aureus in the sample, and wherein the presence of amplified nucleic acid sequence from only elongation factor Tu gene is indicative of the presence of Staphylococcus spp. in said sample.

Thus, the preliminary diagnostic results of the above method are interpreted in the following way: (1) if the test is positive for both Staphylococcus aureus and Staphylococcus spp. (i.e. coagulase negative staphylococci), then the cause of mastitis is likely Staphylococcus aureus, or (2) if the test is negative for Staphylococcus aureus but positive for Staphylococcus spp., then the cause of mastitis is likely Staphylococcus spp. other than Staphylococcus aureus.

In one of the most preferred embodiment of the present invention, said method is performed as a real-time polymerase chain reaction for which several commercial kits are available. Thus, in step b) said isolated nucleic acid obtained in step a) may further be contacted with a probe specifically hybridizing with the nucleic acid sequence of clumping factor gene of Staphylococcus aureus and/or a probe specifically hybridizing with the nucleic acid sequence of elongation factor Tu gene of Staphylococcus spp. The primers of the method can also be used in a multiplex PCR-reaction comprising further primer pairs and probes for simultaneous detection of other bacteria in a milk sample. Such other bacteria may be selected from the group consisting of: Streptococcus dysgalactiae, Streptococcus agalactiae, Streptococcus uberis, Escherichia coli, Enterococcus spp., Corynebacterium bovis, Arcanobacterium pyogenes, Klebsiella spp. (such as Klebsiella pneumoniae and Klebsiella oxytoca), Lactococcus lactis, Serratia spp. (such as Serratia marcescens) and Peptostreptococcus indolicus.

In another preferred embodiment of the present invention, the primers specifically hybridize with the nucleic acid sequence of clumping factor gene of Staphylococcus aureus so that the amplification product obtained comprises at least part of the 95 bp long stretch in said clumping factor gene corresponding to nucleotides 534-628 in SEQ ID NO:8. In elongation factor Tu gene of Staphylococcus spp. the primers preferably amplify at least part of the 119 bp long amplicon in said elongation factor Tu gene corresponding to nucleotides 603-721 in SEQ ID NO:9.

In order to decide the correct treatment for an animal having mastitis, the method of the invention preferably comprises a further or simultaneous step of detecting the presence of β-lactamase production or a β-lactamase gene encoding resistance to β-lactam antibiotics in said sample. If the result of this step is positive, the animal should not be treated with β-lactam antibiotics such as penicillin.

The present invention also provides oligonucleotide primers specifically hybridizing with the nucleic acid sequence of clumping factor gene of Staphylococcus aureus, said primers having the sequence selected from the group consisting of SEQ ID NO:1, SEQ ID NO:2 and SEQ ID NO:3.

The present invention also provides oligonucleotide primers specifically hybridizing with the nucleic acid sequence of elongation factor Tu gene of Staphylococcus spp., said primers having the sequence selected from the group consisting of: SEQ ID NO:4 and SEQ ID NO:5.

For real-time polymerase chain reactions, the invention provides probes specifically hybridizing with the nucleic acid sequence of clumping factor gene of Staphylococcus aureus and with the nucleic acid sequence of elongation factor Tu gene of Staphylococcus spp., said probes having the sequences selected from the group consisting of SEQ ID NO:6 and SEQ ID NO:7, respectively.

The present invention is also directed to the use of a primer or probe specifically hybridizing with the nucleic acid sequence of clumping factor gene of Staphylococcus aureus (and having a sequence as defined in the Sequence Listing) for the detection of the presence of Staphylococcus aureus in a sample.

The present invention is further directed to the use of a primer or probe specifically hybridizing with the nucleic acid sequence of elongation factor Tu gene of Staphylococcus spp. (and having a sequence as defined in the Sequence Listing) for the detection of the presence of Staphylococcus spp. in a sample.

The oligonucleotides of the present invention are short sequences of nucleotides (such as RNA or DNA, preferably DNA), typically with twenty-five to thirty or fewer bases. However, automated synthesizers allow the synthesis of oligonucleotides up to 160 to 200 bases and the present oligonucleotides may be elongated to add, e.g., a restriction enzyme cleavage site, to the oligonucleotide. The typical length of the primers is preferably 18-26, more preferably 20-24 nucleotides.

The present invention also provides kits for the detection of the presence of mastitis causing bacteria in a milk sample. Such a kit may comprise a pair of primers specifically hybridizing with the nucleic acid sequence of clumping factor gene of Staphylococcus aureus and a pair of primers specifically hybridizing with the nucleic acid sequence of elongation factor Tu gene of Staphylococcus spp.

The kit may further comprise a probe specifically hybridizing with the nucleic acid sequence of clumping factor gene of Staphylococcus aureus and/or a probe specifically hybridizing with the nucleic acid sequence of elongation factor Tu gene of Staphylococcus spp. Such primer pairs and probes are described above and in the Examples below.

Preferably, said kit comprises means for a real-time polymerase chain reaction, such as labeled probes, polymerase enzymes, buffers and nucleotides.

It is well-known in the art that the sequences of same gene vary somewhat in strains of a bacterial species and, thus, the sequence of clumping factor gene of Staphylococcus aureus is not 100% identical in all strains of the species. The same applies to the elongation factor Tu gene of Staphylococcus spp. However, it is clear from the description herein, particularly from the Example below, that a person skilled in the art would recognize the sequence of a clumping factor gene of Staphylococcus aureus or a elongation factor Tu gene of Staphylococcus spp. in any related strain based on the similarity and homology of these gene sequences.

Herein the term “specifically hybridizing” means complementary hybridization between an oligonucleotide and a target sequence. The term “specifically” refers to the specificity shown by the complementary hybridization, which allows for minor mismatches between the oligonucleotide and the sequence that may not jeopardize the annealing for detection of hybridization signals.

The publications and other materials used herein to illuminate the background of the invention, and in particular, to provide additional details with respect to its practice, are incorporated herein by reference. The present invention is further described in the following example, which is not intended to limit the scope of the invention.

### Example

### Materials and Methods

### Bacterial Strains

The monoculture bacterial strains used in specificity and sensitivity testing of the oligonucleotide primers and probes contained bacteria from various countries. A total of 424 different samples were tested. The more detailed information about bacterial strains is presented in Table 1.

### Isolation of Template—DNA

Bacterial cells from monocultures were suspended in glycerol. DNA extraction protocol contained three solutions: PPT {69% Trition-X (20%), 9% Tween (100%), 2% ProtK and 20% DTT (200 nM)}, MURLYL {60% mutanolysin (1 U/μl), 15% lysozyme (20 μg/μl), 5% RNAse (20 μg/μl), 20% lysostaphin (1 U/μl) and CHEP II {94% Chelex (50% w/v)}, 6% protK. Equal volumes (100 μl) of bacterial solution and PPT solution was pipetted into a 1.5 mL microcentrifuge tube and vortexed before 5 min incubation at 55° C. The sample was centrifuged for 5 min at 14000 rfc. The supernatant was discarded and bacterial pellet was incubated for 5 min at 95° C. in order to inactivate the protK. The bacterial pellet was suspended in 20 μl of MURLYL solution, and then vortexed and incubated for 30 min at 37° C. After adding 25 μl of CHEP II solution into the tube, the sample was vortexed and incubated for 5 min at 55° C. and then for 5 min at 95° C. After the sample was centrifuged for 5 min at 14000 rfc the upper, DNA containing, bright layer was removed from the tube.

### Oligonucleotide Primers and Probes

S. aureus oligos are designed to amplify 95 bp long amplicon from S. aureus clumping factor gene (clfA). The clfA gene encodes a fibrinogen-binding protein. In Staphylococcus aureus subsp. aureus, strain MRSA252, the coding sequence of clfA having Accession No. BX571856 is as set worth in SEQ ID NO:8. The corresponding gene sequence naturally vary in related strains. In SEQ ID NO:8, said 95 bp long amplicon corresponds to nucleotides 534-628.

Staphylococcus spp. oligos are designed to amplify 119 bp long amplicon from tuf gene, which encodes the elongation factor Tu. In Staphylococcus epidermidis ATCC 12228 the coding sequence of tuf gene having Accession No. NC_{—}004461 is as set worth in SEQ ID NO:9. The corresponding sequence naturally vary in related strains. In SEQ ID NO:9, said 119 bp long amplicon corresponds to nucleotides 603-721.

Tuf and clfA gene oligosequences were obtained from GeneBank (www.ncbi.nih.gov). In addition, four S. aureus strains (173, 2401, 4289, and 5341) were sequenced.

Primers and probes were designed using Primer3 program (available in the internet at http://frodo.wi.mit.edu/cgibin/primer3/primer3_www.cgi). At first, the primers and probes were designed for single qPCR reactions, but then oligos were multiplexed, since their sensitivity and specificity were discovered to be adequately high for multiplex reactions. Designed oligos were divided into three different multiplex primer mixes. Multiplex primer mix 1 contains oligos for S. aureus, C. bovis and Enterococcus spp. detection. Multiplex primer mix 2 contains oligos for Staphylococcus spp., Str. uberis and Str. agalactiae detection, and multiplex primer mix 3 contains oligos for blaZ gene, Str. dysgalactiae and E. coli detection. Designed primer and probe sequences for S. aureus and Staphylococcus spp. detection are disclosed in Table 2.

Multiplex qPCR Assay

The F-450 2× DyNAmo probe Master Mix (Fimmzymes Oy, Espoo, Finland) was used for the multiplex real-time PCR assay. The F-450 2× DyNAmo probe Master Mix contains hot start Thr polymerase, optimized PCR buffer, MgCl₂ and dNTP mix including dUTP. Each reaction contained 10 μl F-450 Master Mix, 500 nM each primer, 250 nM each dual-labeled probe, and sterile water to bring the final volume to 20 μl. The Chromo4 real-time PCR detection system (MJ Research) was used with following program: 95° C. for 10 min followed by 40 repeats of 95° C. for 5 s and 60° C. for 1 min and fluorescence measurement. After 40 cycles repeat samples were incubated 10 s at 10° C.

### Determination of Sensitivity

Sensitivity and specificity of real-time PCR reactions were determined essentially as described in Gillespie and Oliver, 2005.

### Results

While determining the sensitivity and specificity of the S. aureus and Staphylococcus oligos in single qPCR reactions, the threshold line was set to 0.020 (probes contained 6-FAM dye). A sample was determined positive if its fluorescence rose above the threshold line before cycle 35, i.e. its Ct (Cycle threshold) value was under 35. Samples having Ct values over 35 scored negative. Thus, the sensitivities and specificities were 100% in both cases.

Sensitivity for S. aureus oligos: 146/(146+0)*100%=100%

Specificity for S. aureus oligos: 278/(278+0)*100%=100%

Sensitivity for Staphylococcus oligos: 235/(235+0)*100%=100%

Specificity for Staphylococcus oligos: 189/(189+0)*100%=100%

TABLE 1
Sample strains used in specificity and sensitivity testing.

| | | number of |
|---|---|---|
| Bacterial species | Origin of strains | strains |
| S. aureus | Finland, Italy, Norway, Portugal, | 146 |
| | Canada, USA | |
| CNS | Finland, Italy, Norway, Portugal, | 89 |
| | Canada | |
| Str. agalactiae | USA, Canada, Italy, Norway, Portugal | 8 |
| Str. dysgalactiae | USA, Canada, Italy, Norway, Portugal | 31 |
| Str. uberis | Canada, Italy, Norway, Portugal | 26 |
| Str. bovis | Finland | 5 |
| E. coli | Canada, Italy, Norway, Portugal | 45 |
| C. bovis | Portugal | 8 |
| A. pyogenes | Norway | 3 |
| Enterococcus spp. | USA, Italy, Finland | 37 |
| Klebsiella spp. | USA, Canada, Finland | 16 |
| Serratia spp. | Finland, Canada | 5 |
| Pseudomonas spp. | Finland | 1 |
| Citrobacter spp. | Canada | 1 |
| Enterobacter spp. | Finland, Canada | 3 |

TABLE 2

Primer and probe sequences for
S. aureus and Staphylococcus spp.

Primer and probe sequences
S. aureus

| | | |
|---|---|---|
| forward | TTCAACTGAAGCAACACCTTC | SEQ ID NO:1 |
| reverse | CACTTGTATTAACCGCTTGATTAAC | SEQ ID NO:2 |
| reverse2 | TACTTGGATTAACCGCTTGACTAAC | SEQ ID NO:3 |
| probe | TGAATCAGCTCCACAGAGTACAGATGC | SEQ ID NO:6 |

Staphylococcus spp.

| | | |
|---|---|---|
| forward | AACTCCAGAACGTGATTCTGA | SEQ ID NO:4 |
| reverse | TGATTTGACCACGTTCAACAG | SEQ ID NO:5 |
| probe | CATTCATGATGCCAGTTGAGGACGT | SEQ ID NO:7 |

TABLE 3
Amplicons amplified in target strains.

| | |
|---|---|
| S. aureus amplicon in clumping factor gene: | |
| TTCAACTGAAGCAACACCTTCAAACAATGAATCAGCT | SEQ ID NO:10 |
| CCACAGAGTACAGATGCAAGTAATAAAGATGTAGTTA | |
| ATCAAGCGGTTAATACAAGTG | |
| Stafylococcus sp. amplicon in | |
| elongation factor Tu gene: | |
| AACTCCAGAACGTGATTCTGACAAACCATTCATGATG | SEQ ID NO:11 |
| CCAGTTGAGGACGTATTCTCAATCACTGGTCGTGGTA | |
| CTGTTGCTACAGGCCGTGTTGAACGTGGTCAAATCA | |

### REFERENCES

Gillespie, B. E., and S. P. Oliver. 2005. Simultaneous detection of Staphylococcus aureus, Streptococcus agalactiae and Streptococcus uberis in milk by multiplex real-time polymerase chain reaction. J. Dairy Sci. 88:3510-3518.
Maes, N., Magdalena, J., Rottiers, S., De Gheldre, Y., and Struelens M. J. 2002. Evaluation of a Triplex PCR Assay To Discriminate Staphylococcus aureus from Coagulase-Negative Staphylococci and Determine Methicillin Resistance from Blood Cultures. Journal of Clinical Microbiology, 40(4): 1514-1517.
Martineau, F., Picard, F. J., Ke, D., Paradis, S., Roy, P. H., Ouellette, M., and Bergeron, M. G. 2001. Development of a PCR Assay for Identification of Staphylococci at Genus and Species Levels. Journal of Clinical Microbiology, 39(7):2541-2547.
Pitkälä, A., Haveri, M., Pyorala, S., Myllys, V. And Honkanen-Buzalski, T. 2004, Bovine Mastitis in Finland 2001—Prevalence, Distribution of bacteria, and Antimicrobial resistance. J. Dairy Sci. 87:2433-2441
Riffon, R., Sayasith, K., Khalil, H., Dubreuil, P., Drolet, M., and Lagace, J. 2001, Development of a rapid and sensitive test for identification of major pathogens in bovine mastitis by PCR. Journal of Clinical Microbiology 39(7):2584-2589.
Sakai, H., Procop, G. W., Kobayashi, N., Togawa, D., Wilson, D. A., Borden, L., Krebs, V., and Bauer T. W. 2004. Simultaneous Detection of Staphylococcus aureus and Coagulase-Negative Staphylococci in Positive Blood Cultures by Real-Time PCR with Two Fluorescence Resonance Energy Transfer Probe Sets. Journal of Clinical Microbiology, 42(12):5739-5744.
Sandholm, M., Honkanen-Buzalski, T., Kaartinen, L. and Pyörälä, S. 1995, the Bovine Udder and Mastitis. Gummerus Kirjapaino O Y, Jyväskylä.

1. Method for determining the presence of mastitis causing bacteria in a milk sample comprising the steps of:
a) isolating nucleic acid from a milk sample or a sample derived from milk;
b) contacting the isolated nucleic acid obtained in step a) in a polymerase chain reaction mix with primers specifically hybridizing with the nucleic acid sequence of clumping factor gene of Staphylococcus aureus and primers specifically hybridizing with the nucleic acid sequence of elongation factor Tu gene of Staphylococcus spp.; and
c) performing a polymerase chain reaction with a reaction mix obtained from step b) so that the sequences of said clumping factor gene and elongation factor Tu gene are specifically amplified, if said sequences are present in the sample;
d) detecting the presence of the amplified nucleid acid sequences, wherein the presence of amplified nucleic acid sequence from both clumping factor gene and elongation factor Tu gene is indicative of the presence of Staphylococcus aureus in the sample, and wherein the presence of amplified nucleic acid sequence from only elongation factor Tu gene is indicative of the presence of Staphylococcus spp. in the sample.

2. The method according to claim 1, wherein in step b) said isolated nucleic acid obtained in step a) is further contacted with a probe specifically hybridizing with the nucleic acid sequence of clumping factor gene of Staphylococcus aureus and/or a probe specifically hybridizing with the nucleic acid sequence of elongation factor Tu gene of Staphylococcus spp.

3. The method according to claim 2, wherein said polymerase chain reaction mix is for real-time polymerase chain reaction.

4. The method according to claim 3, wherein said real-time polymerase chain reaction is a multiplex reaction.

5. The method according to any one of the preceding claims, wherein the presence of further bacteria is detected in the sample, said bacteria being selected from the group consisting of: Streptococcus dysgalactiae, Streptococcus agalactiae, Streptococcus uberis, Escherichia coli, Enterococcus spp., Corynebacterium bovis, Arcanobacterium pyogenes, Klebsiella spp., Lactococcus lactis, Peptostreptococcus indolicus and Serratia spp.

6. The method according to claim 1, wherein at least one of the primers specifically hybridizing with the nucleic acid sequence of clumping factor gene of Staphylococcus aureus are selected from the sequences consisting of: SEQ ID NO:1, SEQ ID NO:2 and SEQ ID NO:3.

7. The method according to claim 1, wherein at least one of the primers specifically hybridizing with the nucleic acid sequence of elongation factor Tu gene of Staphylococcus spp. are selected from the sequences consisting of: SEQ ID NO:4 and SEQ ID NO:5.

8. The method according to claim 2, wherein the probe specifically hybridizing with the nucleic acid sequence of clumping factor gene of Staphylococcus aureus has the sequence set forth in SEQ ID NO:6.

9. The method according to claim 2, wherein the probe specifically hybridizing with the nucleic acid sequence of elongation factor Tu gene of Staphylococcus spp. has the sequence set forth in SEQ ID NO:7.

10. The method according to claim 1, wherein the presence of β-lactamase production or a β-lactamase gene encoding resistance to β-lactam antibiotics is further detected in said sample.

11. The method according to claim 1, wherein said primers specifically hybridizing with the nucleic acid sequence of clumping factor gene of Staphylococcus aureus amplify at least part of the 95 bp long amplicon in said clumping factor gene corresponding to nucleotides 534-628 in SEQ ID NO:8.

12. The method according to claim 1, wherein said primers specifically hybridizing with the nucleic acid sequence of elongation factor Tu gene of Staphylococcus spp. amplify at least part of the 119 bp long amplicon in said elongation factor Tu gene corresponding to nucleotides 603-721 in SEQ ID NO:9.

13. Use of a primer or probe selected from the group consisting of SED ID NOS:1-4 for the detection of the presence of Staphylococcus aureus in a sample.

14. Use of a primer or probe selected from the group consisting of: SED ID NOS:5-7 for the detection of the presence of Staphylococcus spp. in a sample.

15. An oligonucleotide primer comprising or consisting of a sequence selected from the group consisting of SEQ ID NOS:1-5.

16. An oligonucleotide probe comprising or consisting of a sequence selected from the group consisting of SEQ ID NOS:6 and 7.

17. A kit for the detection of the presence of mastitis causing bacteria in a milk sample comprising a pair of primers specifically hybridizing with the nucleic acid sequence of clumping factor gene of Staphylococcus aureus and a pair of primers specifically hybridizing with the nucleic acid sequence of elongation factor Tu gene of Staphylococcus spp.

18. The kit according to claim 17 further comprising a probe specifically hybridizing with the nucleic acid sequence of clumping factor gene of Staphylococcus aureus and/or a probe specifically hybridizing with the nucleic acid sequence of elongation factor Tu gene of Staphylococcus spp.

19. The kit according to claim 18 for use in the real-time polymerase chain reaction.

20. The kit according to claim 17 or 18, wherein said kit comprises at least one of the primers and probes according to claim 15 or 16.

21. Use of a kit according to claim 17 for the detection of the presence of mastitis causing bacteria in a milk sample.

22. Method for determining the presence of Staphylococcus aureus and Staphylococcus spp. in a sample comprising the steps of:
a) isolating nucleic acid from said sample;
b) contacting the isolated nucleic acid obtained in step a) in a polymerase chain reaction mix with primers specifically hybridizing with the nucleic acid sequence of clumping factor gene of Staphylococcus aureus and primers specifically hybridizing with the nucleic acid sequence of elongation factor Tu gene of Staphylococcus spp.; and
c) performing a polymerase chain reaction with a reaction mix obtained from step b) so that the sequences of said clumping factor gene and elongation factor Tu gene are specifically amplified, if said sequences are present in the sample;
d) detecting the presence of the amplified nucleid acid sequences, wherein the presence of amplified nucleic acid sequence from both clumping factor gene and elongation factor Tu gene is indicative of the presence of Staphylococcus aureus in the sample, and wherein the presence of amplified nucleic acid sequence from only elongation factor Tu gene is indicative of the presence of Staphylococcus spp. in the sample.
